SEQUENCE LISTING (3 sequences)

SEQ ID NO: 1 - DNA, 1282 bp, Erwinia gerundensis
agtaatgtctggggatctgcccgatggagggggataaccactggaaacggtggctaataccgcataacgtcgcaagaccaaagtgggggaccttcgggcctcacaccatcggatgaacccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggggatgaggttaataacctcgttcattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggctgtgagcatgactcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctgctcttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagcagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacac

SEQ ID NO: 2 - DNA, 20 bp, Artificial Sequence
  Primer
agagtttgatcmtggctcag

SEQ ID NO: 3 - DNA, 19 bp, Artificial Sequence
  Primer
ggttaccttgttacgactt

(19) United States
(12) Patent Application Publication
(10) Pub. No.: US 2022/0264891 A1
(43) Pub. Date: Aug. 25, 2022

(54) Novel Erwinia Strains and Related Methods

(71) Applicants: Agriculture Victoria Services PTY LTD, Bundoora, Victoria (AU); Dairy Australia Limited, Southbank, Victoria (AU); Geoffrey Gardiner Dairy Foundation Limited, Melbourne, Victoria (AU)

(72) Inventors: Tongda Li, Southbank (AU); Ian Ross Tannenbaum, Bundoora (AU); Jatinder Kaur, Taylors Hill (AU); Christian Krill, Reservoir (AU); Timothy Ivor Sawbridge, Coburg (AU); Ross Mann, Coburg (AU); German Carlos Spangenberg, Bundoora (AU)

(73) Assignees: Agriculture Victoria Services PTY LTD, Bundoora, Victoria (AU); Dairy Australia Limited, Southbank, Victoria (AU); Geoffrey Gardiner Dairy Foundation Limited, Melbourne, Victoria (AU)

(21) Appl. No.: 17/627,785

(22) PCT Filed: Jul. 17, 2020
(86) PCT No.: PCT/AU2020/050735

§ 371 Date: Jan. 17, 2022

(30) Foreign Application Priority Data
Jul. 19, 2019 (AU) 2019902558

(51) Int. Cl. A01N 63/20; A01H 3/00; C05F 11/08; C12N 1/20

(52) U.S. Cl. CPC A01N 63/20; A01H 3/00; C05F 11/08; C12N 1/205; C12R 2001/18

(57) ABSTRACT

The present invention relates to an endophyte strain isolated from a plant of the Poaceae family, wherein said endophyte is a strain of Erwinia gerundensis which provides bioprotection and/or biofertilizer phenotypes to plants into which it is inoculated. The present invention also discloses plants infected with the endophyte and related methods.

### FIELD OF THE INVENTION

The present invention relates to novel plant microbiome strains, plants infected with such strains and related methods.

### BACKGROUND OF THE INVENTION

Microbes represent an invaluable source of novel genes and compounds that have the potential to be utilised in a range of industrial sectors. Scientific literature gives numerous accounts of microbes being the primary source of antibiotics, immune-suppressants, anticancer agents and cholesterol-lowering drugs, in addition to their use in environmental decontamination and in the production of food and cosmetics.

A relatively unexplored group of microbes known as endophytes, which reside e.g. in the tissues of living plants, offer a particularly diverse source of novel compounds and genes that may provide important benefits to society, and in particular, agriculture.

Endophytes may be fungal or bacterial. Endophytes often form mutualistic relationships with their hosts, with the endophyte conferring increased fitness to the host, often through the production of defence compounds. At the same time, the host plant offers the benefits of a protected environment and nutriment to the endophyte.

Important forage grasses perennial ryegrass (Lolium perenne) are commonly found in association with fungal and bacterial endophytes. However, there remains a general lack of information and knowledge of the endophytes of these grasses as well as of methods for the identification and characterisation of novel endophytes and their deployment in plant improvement programs.

Knowledge of the endophytes of perennial ryegrass may allow certain beneficial traits to be exploited in enhanced pastures, or lead to other agricultural advances, e.g. to the benefit of sustainable agriculture and the environment.

There exists a need to overcome, or at least alleviate, one or more of the difficulties or deficiencies associated with the prior art.

### SUMMARY OF THE INVENTION

In one aspect, the present invention provides a substantially purified or isolated endophyte strain isolated from a plant of the Poaceae family, wherein said endophyte is a strain of Erwinia gerundensis which provides bioprotection and/or biofertilizer phenotypes to plants into which it is inoculated. In a preferred embodiment, the Erwinia gerundensis strain may be strain AR as described herein and as deposited with The National Measurement Institute of 1/153 Bertie Street, Port Melbourne, VIC 3207, Australia on 17 May 2019 with accession number V19/009908.

As used herein the term “endophyte” is meant a bacterial or fungal strain that is closely associated with a plant. By “associated with” in this context is meant that the bacteria or fungus lives on, in or in close proximity to a plant. For example, it may be endophytic, for example living within the internal tissues of a plant, or epiphytic, for example growing externally on a plant.

As used herein the term “substantially purified” is meant that an endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

As used herein the term ‘isolated’ means that an endophyte is removed from its original environment (e.g. the natural environment if it is naturally occurring). For example, a naturally occurring endophyte present in a living plant is not isolated, but the same endophyte separated from some or all of the coexisting materials in the natural system, is isolated.

As used herein the term “bioprotection and/or biofertilizer” means that the endophyte possesses genetic and/or metabolic characteristics that result in a beneficial phenotype in a plant harbouring, or otherwise associated with, the endophyte. Such beneficial properties include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, relative to an organism not harboring the endophyte or harboring a control endophyte such as standard toxic (ST) endophyte.

The pests and/or diseases may include, but are not limited to, fungal and/or bacterial pathogens, preferably fungal. In a particularly preferred embodiment, the endophyte may result in the production of the bioprotectant compound in the plant with which it is associated.

As used herein, the term ‘bioprotectant compound’ is meant as a compound that provides or aids bioprotection to the plant with which it is associated against pests and/or diseases, such as bacterial and/or fungal pathogens. A bioprotectant compound may also be known as a ‘biocidal compound’.

In a particularly preferred embodiment, the endophyte produces a bioprotectant compound and provides bioprotection to the plant against bacterial and/or fungal pathogens. The terms bioprotectant, bioprotective and bioprotection (or any other variations) may be used interchangeably herein.

Thus, in a preferred embodiment, the present invention provides a method of providing bioprotection to a plant against bacterial and/or fungal pathogens, said method including infecting the plant with an endophyte as hereinbefore described and cultivating the plant.

The endophyte may be suitable as a biofertilizer to improve the availability of nutrients to the plant with which the endophyte is associated, including but not limited to improved tolerance to nutrient stress.

Thus, in a preferred embodiment, the present invention provides a method of providing biofertilizer to a plant, said method including infecting the plant with an endophyte as hereinbefore described and cultivating the plant.

The nutrient stress may be lack of or low amounts of a nutrient such as phosphate and/or nitrogen. The endophyte may be capable of growing in conditions such as low nitrogen and/or low phosphate and enable these nutrients to be available to the plant with which the endophyte is associated.

In a preferred embodiment, he endophyte may result in the production of organic acids and/or the solubilisation of phosphate in the organism with which it is associated and/or provide a source of phosphate to the plant.

Alternatively, or in addition, the endophyte may be capable of nitrogen fixation. Thus, if an endophyte is capable of nitrogen fixation, the plant with which the endophyte is associated may be capable of growing in low nitrogen conditions and/or the endophyte may provide a source of nitrogen to the plant.

In a particularly preferred embodiment, the endophyte provides the ability of the organism to grow in low nitrogen.

As used herein the term “plant of the Poaceae family” is a grass species, particularly a pasture grass such as ryegrass (Lolium) or fescue (Festuca), more particularly perennial ryegrass (Lolium perenne L.) or tall fescue (Festuca arundinaceum, otherwise known as Lolium arundinaceum).

In another aspect, the present invention provides a plant or part thereof infected with an endophyte as hereinbefore described. In preferred embodiments, the plant or part thereof infected with the endophyte may produce a bioprotectant compound. Also in preferred embodiments, the plant or part thereof includes an endophyte-free host plant or part thereof stably infected with said endophyte.

The plant inoculated with the endophyte may be a grass or non-grass plant suitable for agriculture, specifically a forage, turf, or bioenergy grass, or a grain crop or industrial crop.

Preferably, the plant is a grass species plant, specifically a forage, turf, bioenergy, grain crop or industrial crop grass.

The forage, turf or bioenergy grass may be those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens] x Brachiaria brizantha, [Brachiaria ruziziensis x Brachiaria brizantha] x Brachiaria decumbens.

The forage, turf or bioenergy grass may also be those belonging to the genera Lolium and Festuca, including L. perenne (perennial ryegrass) and L. arundinaceum (tall fescue) and L. multiflorum (Italian ryegrass).

The grain crop or industrial crop may be a non-grass species, for example, any of soybeans, cotton and grain legumes, such as lentils, field peas, fava beans, lupins and chickpeas, as well as oilseed crops, such as canola.

Thus, the grain crop or industrial crop species may be slected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, and cotton.

The grain crop or industrial crop may be those belonging to the genus Triticum, including T. aestivum (wheat), those belonging to the genus Hordeum, including H. vulgare (barley), those belonging to the genus Avena, including A. sativa (oats), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), and those belonging to the genera Miscanthus, Paspalum, Pennisetum, Poa, Eragrostis and Agrostis.

A plant or part thereof may be infected by a method selected from the group consisting of inoculation, breeding, crossing, hybridisation, transduction, transfection, transformation and/or gene targeting and combinations thereof.

Without wishing to be bound by theory, it is believed that the endophyte of the present invention may be transferred through seed from one plant generation to the next. The endophyte may then spread or locate to other tissues as the plant grows, i.e. to roots. Alternatively, or in addition, the endophyte may be recruited to the plant root, e.g. from soil, and spread or locate to other tissues.

Thus, in a further aspect, the present invention provides a plant, plant seed or other plant part derived from a plant or part thereof as hereinbefore described. In preferred embodiments, the plant, plant seed or other plant part may produce a bioprotectant compound.

In another aspect, the present invention provides the use of an endophyte as hereinbefore described to produce a plant or part thereof stably infected with said endophyte. The present invention also provides the use of an endophyte as hereinbefore described to produce a plant or part thereof as hereinbefore described.

In another aspect, the present invention provides a bioprotectant compound produced by an endophyte as hereinbefore described, or a derivative, isomer and/or a salt thereof.

The bioprotectant compound may be produced by the endophyte when associated with a plant, e.g. a plant of the Poaceae family as described above.

Thus, in another aspect, the present invention provides a method for producing a bioprotectant compound, said method including infecting a plant with an endophyte as hereinbefore described and cultivating the plant under conditions suitable to produce the bioprotectant compound.

The endophyte-infected plant or part thereof may be cultivated by known techniques. The person skilled in the art may readily determine appropriate conditions depending on the plant or part thereof to be cultivated.

The bioprotectant compound may also be produced by the endophyte when it is not associated with a plant. Thus, in yet another aspect, the present invention provides a method for producing a bioprotectant compound, said method including culturing an endophyte as hereinbefore described, under conditions suitable to produce the bioprotectant compound.

The conditions suitable to produce the bioprotectant compound may include a culture medium including a source of carbohydrates. The source of carbohydrates may be a starch/sugar-based agar or broth such as potato dextrose agar, potato dextrose broth or half potato dextrose agar or a cereal-based agar or broth such as oatmeal agar or oatmeal broth. Other sources of carbohydrates may include endophyte agar, Murashige and Skoog with 20% sucrose, half V8 juice/half PDA, water agar and yeast malt extract agar. The endophyte may be cultured under aerobic or anaerobic conditions and may be cultured in a bioreactor.

In a preferred embodiment of this aspect of the invention, the method may include the further step of isolating the bioprotectant compound from the plant or culture medium.

The endophyte-infected plant or part thereof may be cultivated by known techniques. The person skilled in the art may readily determine appropriate conditions depending on the plant or part thereof to be cultivated.

The endophyte of the present invention may be capable of nitrogen fixation. Thus, in yet another aspect, the present invention provides a method of growing the plant in low nitrogen containing medium, said method including infecting a plant with an endophyte as hereinbefore described, and cultivating the plant. Preferably, the low nitrogen medium is low nitrogen containing soil.

In yet a further aspect, the present invention provides a method of increasing nitrogen use efficiency or increasing nitrogen availability to a plant, said method including infecting a plant with an endophyte as hereinbefore described, and cultivating the plant.

In yet another aspect, the present invention provides a method of reducing nitrogen levels in soil, said method including infecting a plant with an endophyte as hereinbefore described, and cultivating the plant in the soil.

In yet another aspect, the present invention provides a method of increasing phosphate use efficiency and/or increasing phosphate solubilisation by a plant, said method including infecting a plant with an endophyte as hereinbefore described, and cultivating the plant.

In yet another aspect, the present invention provides a method of reducing phosphate levels in soil, said method including infecting a plant with an endophyte as hereinbefore described, and cultivating the plant in the soil.

In a further aspect, the present invention provides a method of providing bioprotection to a plant against bacterial and/or fungal pathogens and/or providing biofertilizer to a plant, said method including infecting the plant with and endophyte as hereinbefore described.

Preferably, the method includes providing bioprotection to the plant and includes production of a bioprotectant compound in the plant into which the endophyte is inoculated.

The endophyte-infected plant or part thereof may be cultivated by known techniques. The person skilled in the art may readily determine appropriate conditions depending on the plant or part thereof to be cultivated.

The production of a bioprotectant compound has particular utility in agricultural plant species, in particular, forage, turf, or bioenergy grass species, or grain crop species or industrial crop species. These plants may be cultivated across large areas of e.g. soil where the properties and biological processes of the endophyte as hereinbefore described and/or bioprotectant compound produced by the endophyte may be exploited at scale.

The part thereof of the plant may be, for example, a seed.

In preferred embodiments, the plant is cultivated in the presence of soil phosphate and/or nitrogen, alternatively or in addition to applied phosphate and/or nitrogen. The applied phosphate and/or applied nitrogen may be by way of, for example, fertiliser. Thus, preferably, the plant is cultivated in soil.

In preferred embodiments, the endophyte may be a Erwinia gerundensis strain AR as described herein and as deposited with The National Measurement Institute of 1/153 Bertie Street, Port Melbourne, VIC 3207, Australia on 17 May 2019 with accession number V19/009908.

Preferably, the plant is a forage, turf, bioenergy grass species or, grain crop or industrial crop species, as hereinbefore described.

The part thereof of the plant may be, for example, a seed.

In preferred embodiments, the plant is cultivated in the presence of soil phosphate and/or applied phosphate. The applied phosphate may be by way of, for example, fertiliser. Thus, preferably, the plant is cultivated in soil.

Alternatively, or in addition, the plant is cultivated in the presence of soil nitrogen and/or applied nitrogen. The applied nitrogen may be by way of, for example, fertiliser. Thus, preferably, the plant is cultivated in soil.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

FIGS. 1—16S Amplicon sequence of novel bacterial strain AR (SEQ ID NO. 1).

FIG. 2—Phylogeny of Erwinia spp., Pantoea spp. and novel bacterial strain AR. This maximum-likelihood tree was inferred based on 103 genes conserved among 10 genomes. Values shown next to branches were the local support values calculated using 1000 resamples with the Shimodaira-Hasegawa test.

FIG. 3—Bioprotection bioassay indicating the growth of 11 bacterial stains (including Erwinia gerundensis novel bacterial strain AR, star) against 6 plant pathogenic fungi, Fusarium verticillioides (10 days post inoculation, dpi), Bipolaris gossypina (7 dpi), Sclerotinia rolfsii (5 dpi), Drechslera brizae (8 dpi), Phoma sorghina (9 dpi) and Microdochium nivale (6 dpi). Bars represent the mean diameter of fungal colonies from three replicate plates of each treatment. Different superscript letters indicate significant differences (P<0.05) between treatments.

FIG. 4—Biofertiliser activity (in vitro) of the Erwinia gerundensis novel bacterial strain AR on semi-solid NfB medium. Activity recorded as a change in absorbance at 615 nm over 84 hours (12 hour intervals) relative to absorbance at 615 nm at time 0 hours. The Erwinia gerundensis novel bacterial strain AR was compared to an Escherichia coli negative control strain, and a no growth control (NGC—NfB media only).

FIG. 5—Image of 5 day old seedlings inoculated with the Erwinia gerundensis novel bacterial strain AR and an untreated control.

FIG. 6—Average shoot and root length of barley seedlings inoculated with the Erwinia gerundensis novel bacterial strain AR and an untreated control (blank), and grown for 5 days. The root length was significantly different (p-value <0.05) between the two treatments, but not the shoot length.

FIG. 7—Average root length of barley seedlings inoculated with bacterial strains of Erwinia gerundensis (strain AR) and non-Erwinia strains (Strain 1, 2, 3, 4) and grown for 4 days on nitrogen free media. The star indicates significant difference in the mean at p 0.05 between the control and the bacterial strains.

FIG. 8—Average shoot length of barley seedlings inoculated with bacterial strains of Erwinia gerundensis (strain AR) and non-Erwinia strains (Strain 1, 2, 3, 4) and grown for 4 days on nitrogen free media. The star indicates significant difference in the mean at p 0.05 between the control and the bacterial strains.

### DETAILED DESCRIPTION OF THE EMBODIMENTS

Isolation and Characterisation of Plant Associated Erwinia gerundensis Novel Bacterial Strains Providing Bioprotection and Biofertilizer Phenotypes to Plants.

The novel plant associated Erwinia gerundensis bacterial strain AR has been isolated from perennial ryegrass (Lolium perenne) plants. It displays the ability to inhibit the growth of plant fungal pathogens, grow under low N conditions in plate assays, and have some plant growth promotion abilities. The genome of the Erwinia gerundensis novel bacterial strain AR has been sequenced and is shown to be novel, related to the species Erwinia gerundensis. This novel bacterial strain has been used to inoculate barley (Hordeum vulgare) seeds under glasshouse conditions and has been demonstrated not to cause disease in these barley plants. These barley plants are also able to produce seed. Novel bacterial strain AR also enhances root and shoot growth in nitrogen limiting conditions Overall, novel plant associated Erwinia gerundensis novel bacterial strain AR offer both bioprotectant and biofertilizer activity.

### Example 1—Isolation of Bacterial Strains

Seed Associated Bacterial Strains

Seeds from perennial ryegrass (Lolium perenne) were surface-sterilised by soaking in 80% ethanol for 3 mins, then washing 5 times in sterile distilled water. The seeds were then plated onto sterile filter paper soaked in sterile water in sterile petri dishes. These plates were stored at room temperature in the dark to allow seedlings to germinate for 1-2 weeks. Once the seedlings were of sufficient size, the plants were harvested. In harvesting, the remaining seed coat was discarded, and the aerial tissue and root tissue were harvested. The plant tissues were submerged in sufficient Phosphate Buffered Saline (PBS) to completely cover the tissue, and ground using a Qiagen TissueLyser II, for 1 minute at 30 Hertz. A 10 μl aliquot of the macerate was added to 90 μl of PBS. Subsequent 1 in 10 dilutions of the 10⁻¹ suspension were used to create additional 10⁻² to 10⁻⁴ suspensions. Once the suspensions were well mixed 50 μl aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony.

Mature Plant Associated Bacterial Strains

Leaf and root tissue were harvested from mature plants grown in the field or grown in pots in a greenhouse. Root tissue was washed in PBS buffer to remove soil particles and sonicated (10 mins) to remove the rhizosphere. The harvested tissues were placed into sufficient PBS to completely cover the tissue and processed as per the previous section to isolate pure bacterial cultures.

Around 300 bacterial strains were obtained from sterile seedlings, and 300 strains from mature plants. The novel bacterial strain AR was collected from seed of perennial ryegrass.

### Example 2—Identification of Erwinia gerundensis Novel Bacterial Strain

Amplicon (16S rRNA Gene) Sequencing

A phylogenetic analysis of the novel bacterial strain AR was undertaken by sequence homology comparison of the 16S rRNA gene. The novel bacterial strain AR was grown overnight in Reasoners 2 Broth (R2B) media. DNA was extracted from pellets derived from the overnight culture using a DNeasy Blood and Tissue kit (Qiagen) according to manufacturer's instructions. The 16S rRNA gene amplification used the following PCR reagents: 14.8 μL H₂O, 2.5 μL 10× reaction buffer, 0.5 μL 10 mM dNTPs, 2.5 μL each of the 5 μM 27F primer (5′-AGAGTTTGATCMTGGCTCAG-3′) (SEQ ID NO: 2) and 5 μM reverse primers 1492R (5′-GGTTACCTTGTTACGACTT-3′) (SEQ ID NO. 3), 0.2 μL of Immolase enzyme, and template to a final volume of 25 μL. The PCR reaction was then run in an Agilent Surecycler 8800 (Applied Biosystems) with the following program; a denaturation step at 94° C. for 15 min; 35 cycles of 94° C. for 30 sec, 55° C. for 10 sec, 72° C. 1 min; and a final extension step at 72° C. for 10 min.

Shrimp alkaline phosphatase (SAP) exonuclease was used to purify the 16S rRNA gene PCR amplicon. The SAP amplicon purification used the following reagents: 7.375 μL H₂O, 2.5 μL 10×SAP, and 0.125 μL Exonuclease I. The purification reaction was incubated at 37° C. for 1 hr, followed by 15 min at 80° C. to deactivate the exonuclease.

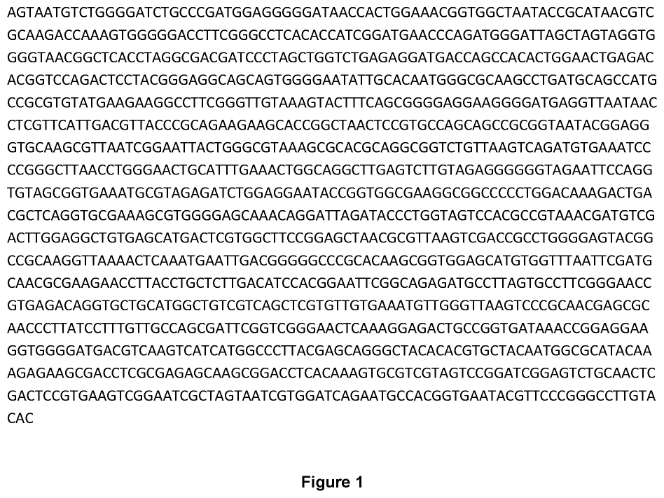

The purified 16S rRNA gene amplicon was sequenced using the BigDye® Terminator v3.1 Cycle Sequencing Kit (Thermofisher) with the following reagents; 10.5 μL H₂O, 3.5 μL 5×Seq buffer, 0.5 μL BigDye®, 2.5 μL of either the 3.2 μM Forward (27F) and 3.2 μM Reverse primers (1492R), and 4.5 μL of PCR amplicon as template, to a final reaction volume of 20 μL. The sequencing PCR reaction was then run in an Agilent Surecycler 8800 (Applied Biosystems) with the following program; denaturation step at 94° C. for 15 min; followed by 35 cycles of 94° C. for 30 sec, 55° C. for 10 sec, 72° C. 1 min; and one final extension step at 72° C. for 10 min. The 16S rRNA gene amplicon from novel bacterial strain AR was sequenced on an AB13730XL (Applied Biosystems). A 1282 bp 16S rRNA gene sequence was generated (FIG. 1; SEQ ID NO. 1). The sequence was aligned by BLASTn on NCBI against the non-redundant nucleotide database and the 16S ribosomal RNA database.

BLASTn Hit Against Database Nr; Erwinia sp. Strain KUDC3014 16S Ribosomal RNA Gene, Partial Sequence

| Max | Total | Query | E- | | |
|---|---|---|---|---|---|
| Score | Score | Coverage | Value | % Identity | Accession |
| 2313 | 2313 | 100% | 0 | 100.00% | MK070133.1 |

BLASTn Hit Against Database 16S Ribosomal RNA; Erwinia gerundensis Strain EM595 16S Ribosomal RNA Gene, Partial Sequence

| Max | Total | Query | E- | | |
|---|---|---|---|---|---|
| Score | Score | Coverage | Value | % Identity | Accession |
| 2264 | 2264 | 100% | 0 | 98.91% | NR_148820.1 |

The preliminary taxonomic identification of the novel bacterial strain AR was Erwinia gerundensis.

Genomics

The genome of novel bacterial strain AR was sequenced. This novel bacterial strain was retrieved from the glycerol collection stored at −80° C. by streaking on R2A plates. Single colonies from these plates were grown overnight in Nutrient Broth and pelleted. These pellets were used for genomic DNA extraction using the bacteria protocol of Wizard® Genomic DNA Purification Kit (A1120, Promega). A DNA sequencing library was generated for Illumina sequencing using the Illumina Nextera XT DNA library prep protocol. The library was sequenced using an Illumina MiSeq platform or HiSeq platform. Raw reads from the sequencer were filtered to remove any adapter and index sequences as well as low quality bases using Trimmomatic (Bolger, Lohse & Usadel 2014) with the following options: ILLUMINACLIP: NexteraPE-PE.fa:2:30:10 LEADING:3 TRAILING:3 SLIDINGWINDOW:4:15 MINLEN:36. To enable full genome assembly, long reads were generated for novel bacterial strain AR only by sequencing DNA using Oxford Nanopore Technologies (ONT) MinION platform. The DNA from the Wizard® Genomic DNA Purification Kit was first assessed with the genomic assay on Agilent 2200 TapeStation system (Agilent Technologies, Santa Clara, Calif., USA) for integrity (average molecular weight 30 Kb). The sequencing library was prepared using an in-house protocol modified from the official protocols for transposases-based library preparation kits (SQK-RAD004/SQK-RBK004, ONT, Oxford, UK). The library was sequenced on a MinION Mk1B platform (MIN-101B) with R9.4 flow cells (FLO-MIN106) and under the control of MinKNOW software. After the sequencing run finished, the fast5 files that contain raw read signals were transferred to a separate, high performance computing Linux server for local basecalling using ONT's Albacore software (Version 2.3.1) with default parameters. The sequencing summary file produced by Albacore was processed by the R script minion qc (https://github.com/roblanf/minion_qc) and NanoPlot (De Coster et al. 2018) to assess the quality of the sequencing run, while Porechop (Version 0.2.3, https://github.com/rrwick/Porechop) was used to remove adapter sequences from the reads. Reads which were shorter than 300 bp were removed and the worst 5% of reads (based on quality) were discarded by using Filtlong (Version 0.2.0, https://github.com/rrwick/Filtlong).

The whole genome sequence of novel bacterial strain AR was assembled using Unicycler (Wick et al. 2017). Unicycler performed hybrid assembly when both Illumina reads and MinION reads were available. MinION reads were mainly used to resolve repeat regions in the genome, whereas Illumina reads were used by Pilon (Walker et al. 2014) to correct small base-level errors. Multiple rounds of Racon (Vaser et al. 2017) polishing were then carried out to generate consensus sequences. Assembly graphs were visualised by using Bandage (Wick et al. 2015).

A complete circular chromosome sequence and two plasmid sequences were produced for the Erwinia gerundensis novel bacterial strain AR. The genome size for the novel bacterial strain AR was 3,748,909 bp (Table 1). The percent GC content ranged from 55% to 53% for the genome and plasmids. The novel bacterial strain AR was annotated by Prokka (Seemann 2014) with a custom, genus-specific protein database to predict genes and corresponding functions, which were then screened manually to identify specific traits. The number of genes for the novel bacterial strain AR was 4,091 (Table 2).

TABLE 1
Summary of properties of the final genome sequence assembly

| | | GC | Coverage | Coverage |
|---|---|---|---|---|
| | Genome | content | Illumina | ONT |
| Strain ID | size (bp) | (%) | reads | MinION |
| AR chromosome | 3,748,909 | 55 | 150× | 150× |
| AR plasmid 1 | 580,656 | 55 | 150× | 150× |
| AR plasmid 2 | 107,871 | 53 | 150× | 150× |

TABLE 2
Summary of genome coding regions

| Strain | Genome size | No. of | No. of | No. of | No. of | No. of |
|---|---|---|---|---|---|---|
| ID | (bp) | tRNA | tmRNA | rRNA | CDS | gene |
| AR | 4,437,426 | 78 | 0 | 22 | 3,991 | 4,091 |

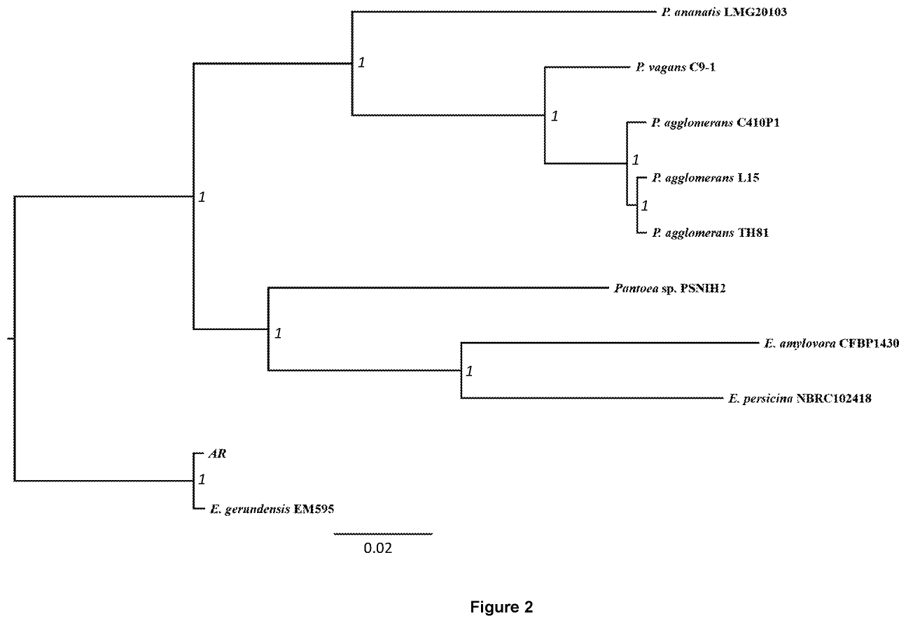

Nine Erwinia and Pantoea spp. (P. sp PSNIH2, P. ananatis LMG20103, P. vagans C9-1, P. agglomerans C410P1, L15 and TH81, E. amylovora CFBP1430, E. persicina NBRC102418, and E. gerundensis EM595) genome sequences that are publicly available on NCBI were acquired and used for pan-genome/comparative genome sequence analysis alongside the novel bacterial strain AR (E. gurendensis). A total of 103 genes that are shared by all 10 bacterial strains were identified by running Roary (Page et al. 2015). PRANK (Löytynoja 2014) was then used to perform a codon aware alignment. A maximum-likelihood phylogenetic tree (FIG. 2) was inferred using FastTree (Price, Dehal & Arkin 2010) with Jukes-Cantor Joins distances and Generalized Time-Reversible and CAT approximation model. Local support values for branches were calculated using 1000 resamples with the Shimodaira-Hasegawa test. The novel bacterial strain AR clustered tightly with the Erwinia gerundensis bacterial strain EM595, suggesting a close phylogenetic relationship between these two bacterial strains. Moreover, this cluster was separated from other Pantoea and Erwinia spp. with strong local support value (100%). This separation supports that bacterial strain AR is novel and from the species Erwinia gerundensis.

### Example 3—Bioprotection Activity (In Vitro) of the Erwinia gerundensis Novel Bacterial Strain AR

In vitro bioassays were established to test the bioactivity of the Erwinia gerundensis novel bacterial strain AR, against six plant pathogenic fungi (Table 3). A plate with only the pathogen was used as a negative control (blank). The fungal pathogens were all isolated from monocot species, and were obtained from the National Collection of Fungi (Herbarium VPRI) and the AVR collection. Each bacterial strain was cultured in Nutrient Broth (BD Biosciences) overnight at 28° C. in a shaking incubator (200 rpm). Each bacterial strain was drop-inoculated (20 μL) onto four equidistant points on a Nutrient Agar (BD Biosciences) plate, which was then incubated overnight at 28° C. A 6 mm×6 mm agar plug of actively growing mycelia from the pathogen was placed at the centre of the plate. The bioassay was incubated for at least 5 days at 28° C. in the dark, and then the diameter of the fungal colony on the plate was recorded. For each treatment three plates were prepared as biological triplicates. OriginPro 2018 (Version b9.5.1.195) was used to carry out One-way ANOVA and Tukey Test to detect the presence of any significant difference (p≤0.05) between treatments.

TABLE 3
Pathogens used in the bioprotection bioassay.

| VPRI | | Host | | |
|---|---|---|---|---|
| Accession | | Taxonomic | | Collection |
| No. | Taxonomic Details | Details | State | Date |
| 12962 | Drechslera brizae | Briza | Vic. | 24 Oct. 1985 |
| | (Y. Nisik.) Subram. & | maxima L. | | |
| | B.L. Jain | | | |
| 32148 | Sclerotium rolfsii Sacc. | Poa annua L. | Vic. | 1 Jan. 2005 |
| 10694 | Phoma sorghina (Sacc.) | Cynodon | Vic. | 19 Apr. 1979 |
| | Boerema, Dorenbosch, | dactylon Pers. | | |
| | van Kesteren | | | |
| 42586a | Fusarium verticillioides | Zea mays L. | Vic. | 27 Feb. 2015 |
| | (Sacc.) Nirenberg | | | |
| 42563 | Bipolaris gossypina | Brachiaria | Qld | |
| N/A | Microdochium nivale | Lolium | Vic | |
| | | perenne L. | | |

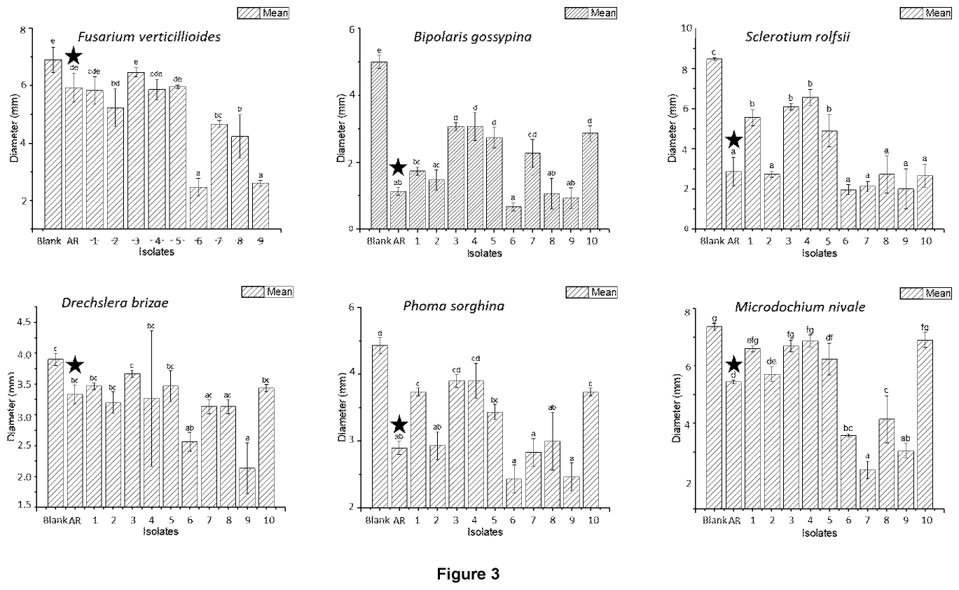

The Erwinia gerundensis novel bacterial strain AR inhibited the growth of four of the six fungal pathogens compared to the control (FIG. 3). The Erwinia gerundensis novel bacterial strain AR was active against Bipolaris gossypina, Sclerotium rolfsii and Phoma sorghina, and Microdochium nivale.

### Example 4—Biofertiliser Activity (In Vitro) of the Erwinia gerundensis Novel Bacterial Strain AR

Nitrogen (N) is an important nutrient for plant growth and a key component of fertilisers. Plant associated bacteria able to grow under low nitrogen conditions may be useful in plant growth as they can pass this N onto the plant. The ability to grow under low nitrogen conditions was assessed by using the nitrogen-free NFb medium (Dobereiner 1980) and Burks medium (Wilson & Knight 1952). One litre of NFb medium contains 5 g DL-malic acid, 0.5 g dipotassium hydrogen orthophosphate, 0.2 g magnesium sulfate heptahydrate, 0.1 g sodium chloride, 0.02 g calcium chloride dehydrate, 2 mL micronutrients solution [0.4 g/L copper sulfate pentahydrate, 0.12 g/L zinc sulfate heptahydrate, 1.4 g/L boric acid, 1 g/L sodium molybdate dehydrate, 1.5 g/L manganese(II) sulfate monohydrate], 1 mL vitamin solution (0.1 g/L biotin, 0.2 g/L pyridoxol hydrochloride), 4 mL iron(III) EDTA and 2 mL bromothymol blue (0.5%, dissolved in 0.2N potassium hydroxide). For solid NFb medium, 15 g/L bacteriological agar was added, otherwise 0.5 g/L was added for semi-solid medium. The pH of medium was adjusted to 6.8. The contents of Burks medium include 10 g/L dextrose, 0.41 g/L potassium dihydrogen phosphate, 0.52 g/L dipotassium hydrogen orthophosphate, 0.05 g/L sodium sulfate, 0.2 g/L calcium chloride, 0.1 g/L magnesium sulfate heptahydrate, 0.005 g/L iron(II) sulfate heptahydrate, 0.0025 g/L sodium molybdate dehydrate and 15 g/L bacteriological agar. The pH of medium was adjusted to 7. To detect the nitrogen fixation ability, bacterial strains, including E. coli as a negative control, were inoculated onto solid medium plates. For each inoculation, triplicates were prepared. All NFb medium plates were incubated at 30° C., whereas Burks medium plates were incubated at 28° C. After 96 hours, the colour change of NFb medium plates was recorded, with development of blue colour an indication of growth under limiting N. The physical growth of bacteria on Burks medium plates was the indicator for this assay. To evaluate if the nitrogen is the limiting factor in Burks medium, a control group whose Burks medium was supplemented with 10 g/L tryptone or ammonia chloride was added to the bioassay.

In the high throughput automated method to detect nitrogen fixation ability semi-solid media NfB was used. Bacterial strains (including E. coli negative control) were inoculated into 20 mL R2B medium (0.5 g/L yeast extract, 0.5 g/L proteose peptone, 0.5 g/L casein hydrolysate, 0.5 g/L glucose, 0.5 g/L starch, 0.3 g/L dipotassium hydrogen orthophosphate, 0.024 g/L magnesium sulphate and 0.3 g/L sodium pyruvate) and incubated at 28° C. and 200 rpm overnight. The cell pellet was collected by centrifuging at 4000×g for 3 minutes, and then was twice with 1×PBS to remove the nitrogen residue from R2B. Then cell pellet was resuspended in 10 mL semi-solid NFb medium. 1 μL of cell suspension was added to a well containing 199 μL semi-solid NFb medium on a 96-well cell culture plate. For each bacterial strain, the cell suspension was added to six consecutive wells of the same column, representing six biological replicates. After inoculating all bacterial strains, the plate was examined by plate reader immediately by obtaining a reading at 615 nm wavelength. Wells located in rows A and H, and columns 1 and 12 were excluded during the examination due to the edge effect which may lead to an unreliable reading. The plate was incubated at room temperature for 84 hours, during which it was examined by plate reader every 12 hours. Values were expressed as differences in absorbance at 615 nm relative to the absorbance at 615 nm in the well at time zero. An increase in absorbance represented an increase in growth under low nitrogen conditions.

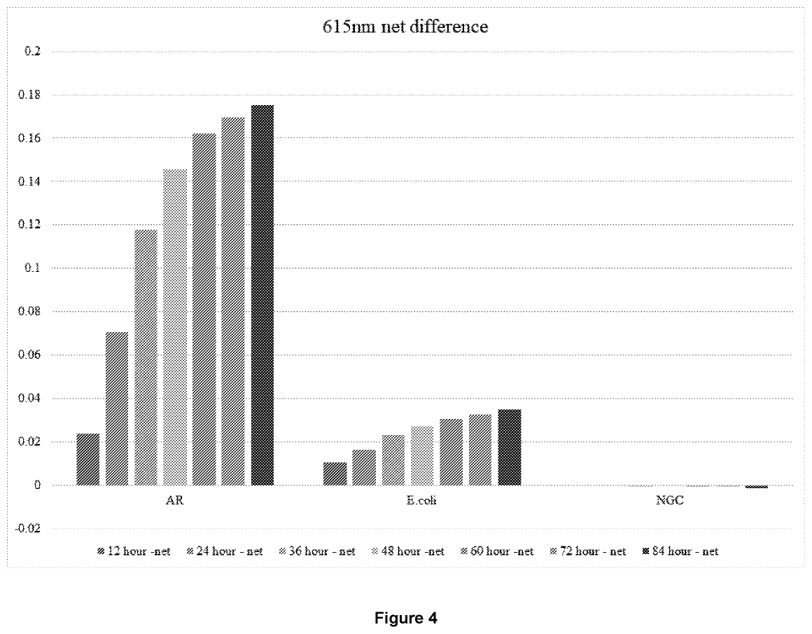

The Erwinia gerundensis novel bacterial strain AR was able to grow under low N, as evident from the colour change in the NfB media, growth on Burks media (without supplementary N source) and elevated absorbance levels at a wavelength of 615 nm in comparison to the E. coli negative control and no growth control (NfB media only) (FIG. 4).

### Example 5—in Planta Inoculations Supporting Endophytic Niche of the Erwinia gerundensis Novel Bacterial Strain AR

To assess direct interactions between the Erwinia gerundensis novel bacterial strain AR and plants, an early seedling growth assay was established in barley. The Erwinia gerundensis novel bacterial strain AR was cultured in Lysogeny Broth (LB) overnight at 26° C. The following day seeds of barley (cultivar Hindmarsh) were surface-sterilised by soaking in 80% ethanol for 3 mins, then washing 5 times in sterile distilled water. The seeds were then soaked in the overnight cultures for 4 hours at 26° C. in a shaking incubator. For control seedlings, seeds were soaked in LB without bacteria for 4 hours at 26° C. in a shaking incubator. The seeds were planted into a pot trial, with three replicates (pots) per strain/control, with a randomised design. A total of 20 seeds were planted per pot, to a depth of 1 cm. The potting medium contained a mixture of 25% potting mix, 37.5% vermiculite and 37.5% perlite. The plants were grown for 5 days and then removed from the pots, washed, assessed for health (i.e. no disease symptoms) and photographed. The lengths of the longest root and the longest shoot were measured. Data was statistically analysed using a t test to detect the presence of any significant difference (p≤0.05) between treatments using Excel.

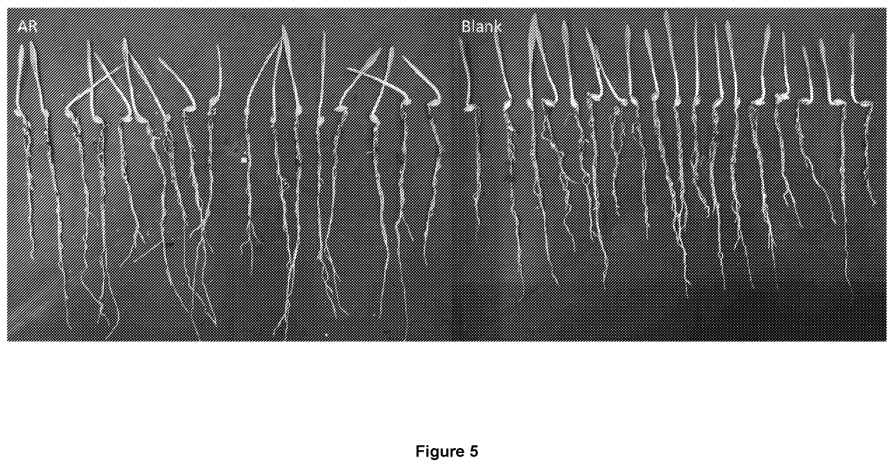

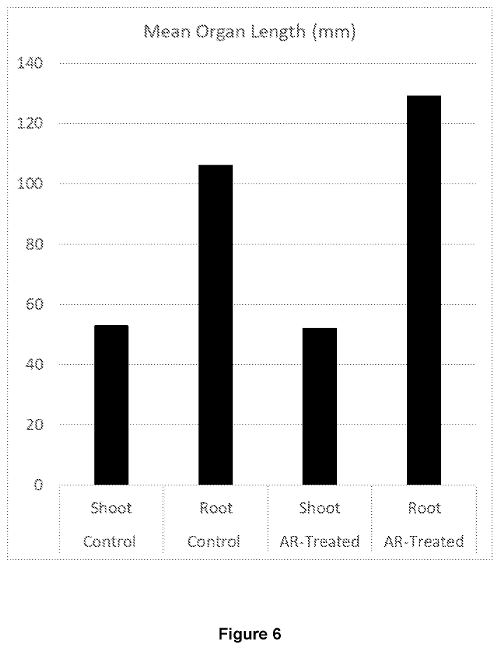

Seedlings inoculated with the Erwinia gerundensis novel bacterial strain AR were healthy with no disease symptoms recorded on leaves or roots (FIG. 5). The mean length of the shoots inoculated with the Erwinia gerundensis novel bacterial strain AR were equivalent to the control (FIG. 6) at 53.5 to 54.4 mm. The length of the roots of seedlings inoculated with the Erwinia gerundensis novel bacterial strain AR were significantly longer than the control (FIG. 6) at 131.2 mm to 107.7 mm (T-test 0.001675013).

### Example 6—in Planta Inoculations Supporting the Biofertilizer (Nitrogen) Niche of the Erwinia gerundensis Novel Bacterial Strain AR

An in planta biofertilizer assay was established in barley to evaluate the ability of Erwinia gerundensis novel bacterial strain AR to aid growth under nitrogen limiting conditions. Initially, bacterial strains (5, including AR were cultured in 20 mL nutrient broth (BD Bioscience) overnight at 26° C. whilst rotating at 200 RPM. The following day cultures were pelleted via centrifugation at 4000 RPM for 5 minutes, washed three times in 10 mL Phosphate Buffered Saline (PBS), resuspended in 20 mL PBS, quantified via spectrophotometry (OD600) and diluted (1:10). Barley seeds were sterilized in 70% ethanol for 5 minutes, followed by rinsing with sterilized distilled water (SDW) for five times. These sterile seeds were submerged in the dilution for 4 hours in a dark incubator at room temperature whilst rotating at 200 RPM. The seeds were subsequently transferred to moistened sterile filter paper and allowed to germinate for three days. The three-day-old seedlings were individually transferred to 60 mm plates with semi-solid Burks media (HiMedia) (5 g/L Agar). Seedlings were allowed to grow for a further 4 days, before the shoots and roots were measured for each seedling. There was a total of 6 treatments (5 bacterial strains including AR; 1 blank media control) containing 10 seedlings per treatment. Statistical analysis (One-way ANOVA and Tukey Test) was conducted using OriginPro 2018 (Version b9.5.1.195) to detect the presence of any significant difference (P<0.05) between treatments.

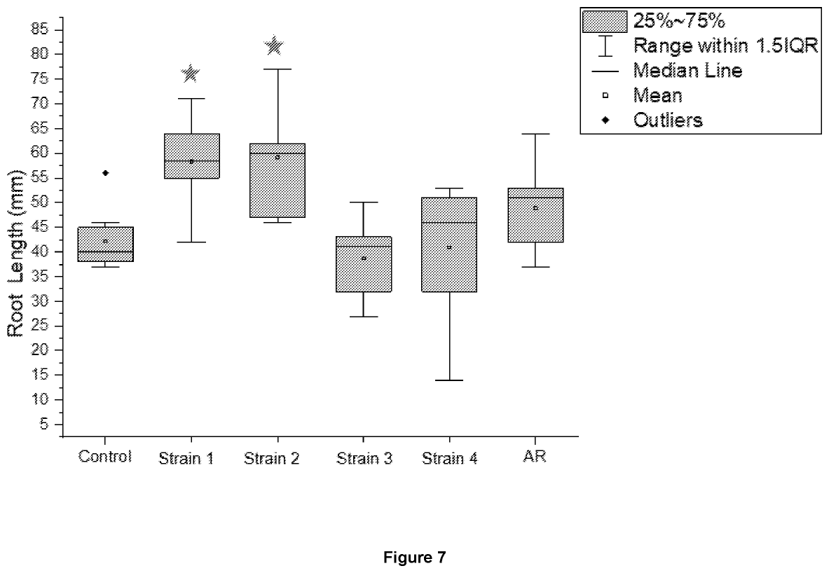

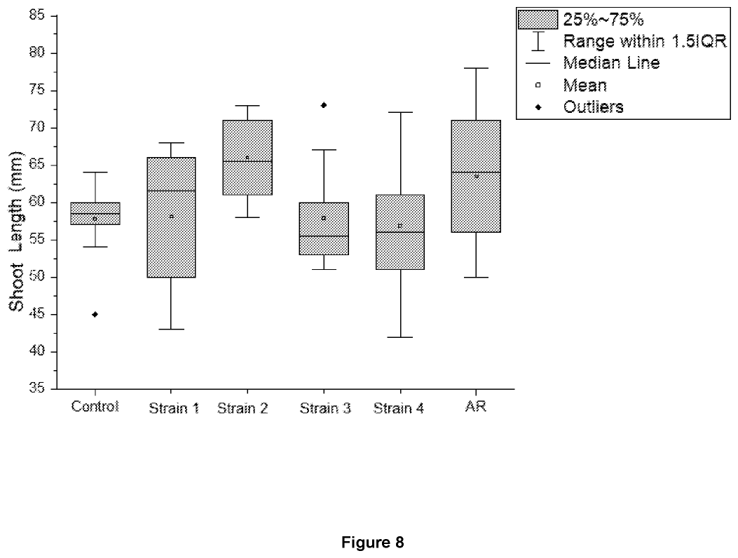

The root growth of seedlings inoculated with novel bacterial strain AR and grown under nitrogen limiting conditions was not significantly greater than the control (P<0.05), despite increasing root growth by 13.6% (FIG. 7). The shoot growth of seedlings inoculated with novel bacterial strain AR was not significantly greater than the control (P<0.05), despite increasing shoot growth by 9.0% (FIG. 8). Overall, results indicate that novel bacterial strain AR can aid in the growth of seedlings grown under nitrogen limiting conditions.

It is to be understood that various alterations, modifications and/or additions may be made without departing from the spirit of the present invention as outlined herein.

As used herein, except where the context requires otherwise, the term “comprise” and variations of the term, such as “comprising”, “comprises” and “comprised”, are not intended to be in any way limiting or to exclude further additives, components, integers or steps.

Reference to any prior art in the specification is not, and should not be taken as, an acknowledgment or any form of suggestion that this prior art forms part of the common general knowledge in Australia or any other jurisdiction or that this prior art could reasonably be expected to be combined by a person skilled in the art.

### REFERENCES

1. Bolger, A M, Lohse, M & Usadel, B 2014, ‘Trimmomatic: a flexible trimmer for Illumina sequence data’, Bioinformatics, vol. 30, no. 15, pp. 2114-20.
2. De Coster, W, D'Hert, S, Schultz, D T, Cruts, M & Van Broeckhoven, C 2018, ‘NanoPack: visualizing and processing long read sequencing data’, Bioinformatics.
3. Dobereiner, J 1980, ‘Forage grasses and grain crops’, in Methods for evaluating biological nitrogen fixation/edited by F J Bergersen, John Wiley & Sons Ltd, pp. 535-55.
4. Löytynoja, A 2014, ‘Phylogeny-aware alignment with PRANK’, in D J Russell (ed.), Multiple Sequence Alignment Methods, Humana Press, Totowa, N.J., pp. 155-70, DOI 10.1007/978-1-62703-646-7_10, <https://doi.org/10.1007/978-1-62703-646-7_10>.
5. Page, A J, Cummins, C A, Hunt, M, Wong, V K, Reuter, S, Holden, M T, Fookes, M, Falush, D, Keane, J A & Parkhill, J 2015, ‘Roary: rapid large-scale prokaryote pan genome analysis’, Bioinformatics, vol. 31, no. 22, pp. 3691-3.
6. Price, M N, Dehal, P S & Arkin, A P 2010, ‘FastTree 2—approximately maximum-likelihood trees for large alignments’, PLoS One, vol. 5, no. 3, p. e9490.
7. Seemann, T 2014, ‘Prokka: rapid prokaryotic genome annotation’, Bioinformatics, vol. 30, no. 14, pp. 2068-9.
8. Vaser, R, Sovic, I, Nagarajan, N & Sikic, M 2017, ‘Fast and accurate de novo genome assembly from long uncorrected reads’, Genome Res, vol. 27, no. 5, pp. 737-46.
9. Walker, B J, Abeel, T, Shea, T, Priest, M, Abouelliel, A, Sakthikumar, S, Cuomo, C A, Zeng, Q, Wortman, J, Young, S K & Earl, A M 2014, ‘Pilon: an integrated tool for comprehensive microbial variant detection and genome assembly improvement’, PLoS One, vol. 9, no. 11, p. e112963.
10. Wick, R R, Judd, L M, Gorrie, C L & Holt, K E 2017, ‘Unicycler: Resolving bacterial genome assemblies from short and long sequencing reads’, PLOS Computational Biology, vol. 13, no. 6, p. e1005595.
11. Wick, R R, Schultz, M B, Zobel, J & Holt, K E 2015, ‘Bandage: interactive visualization of de novo genome assemblies’, Bioinformatics, vol. 31, no. 20, pp. 3350-2.
12. Wilson, P W & Knight, S G 1952, Experiments in Bacterial Physiology, Burgess.

1-25. (canceled)

26. A substantially purified or isolated endophyte strain isolated from a plant of the Poaceae family, wherein said endophyte is a strain of Erwinia gerundensis which provides bioprotection and/or biofertilizer phenotypes to plants into which it is inoculated.

27. The endophyte according to claim 26, wherein the bioprotection and/or biofertilizer phenotype includes production of a bioprotectant compound in the plant into which the endophyte is inoculated.

28. The endophyte according to claim 26, wherein the bioprotection and/or biofertilizer phenotype includes nitrogen fixation in the plant into which the endophyte is inoculated.

29. The endophyte according to claim 26, wherein the endophyte is Erwinia gerundensis strain AR as deposited with The National Measurement Institute on 17 May 2019 with accession number V19/009908.

30. The endophyte according to claim 26, wherein the plant from which the endophyte is isolated is a pasture grass.

31. The endophyte according to claim 30, wherein the pasture grass is from the genus Lolium or Festuca.

32. The endophyte according to claim 31, wherein the pasture grass is from the species Lolium perenne or Festuca arundinaceum.

33. The endophyte according to claim 26, wherein the plant into which the endophyte is inoculated includes an endophyte-free host plant or part thereof stably infected with said endophyte.

34. The endophyte according to claim 26, wherein the plant into which the endophyte is inoculated is an agricultural plant selected from one or more of forage grass, turf grass, bioenergy grass, grain crop and industrial crop.

35. The endophyte according claim 34, wherein the plant into which the endophyte is inoculated is a forage, turf or bioenergy grass selected from the group consisting of those belonging to the genera Lolium and Festuca, including L. perenne (perennial ryegrass), L. arundinaceum (tall fescue) and L. multiflorum (Italian ryegrass), and those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens]x Brachiaria brizantha, [Brachiaria ruziziensis x Brachiaria brizantha] x Brachiaria decumbens; or
wherein the plant into which the endophyte is inoculated is a grain crop or industrial crop grass selected from the group consisting of those belonging to the genus Triticum, including T aestivum (wheat), those belonging to the genus Hordeum, including H. vulgare (barley), those belonging to the genus Avena, including A. sativa (oats), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), those belonging to the genera Miscanthus, Paspalum, Pennisetum, Poa, Eragrostis and Agrostis; or
wherein the plant into which the endophyte is inoculated is a grain crop or industrial crop selected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, and cotton.

36. A plant or part thereof infected with one or more endophyte according to claim 26.

37. A bioprotectant compound produced by the endophyte according to claim 26, or a derivative, isomer and/or a salt thereof.

38. A method for producing a bioprotectant compound, or a derivative, isomer and/or a salt thereof, said method including infecting a plant with the endophyte according to claim 26 and cultivating the plant under conditions suitable to produce the bioprotectant compound.

39. A method for producing a bioprotectant compound, or a derivative, isomer and/or a salt thereof, said method including culturing the endophyte according to claim 26 under conditions suitable to produce the bioprotectant compound.

40. A method according to claim 39, wherein the conditions include a culture medium including a source of carbohydrates, preferably wherein the source of carbohydrates is selected from one or more of the group consisting of a starch/sugar-based agar or broth, a cereal-based agar or broth, endophyte agar, Murashige and Skoog with 20% sucrose, half V8 juice/half PDA, water agar and yeast malt extract agar.

41. The method according to claim 39, wherein the method further includes isolating the bioprotectant compound from the plant or culture medium.

42. A method of providing bioprotection to a plant against bacterial and/or fungal pathogens and/or providing biofertilizer to a plant, said method including infecting the plant with the endophyte according to claim 26 and cultivating the plant.

43. The method according to claim 42, wherein the method includes increasing nitrogen use efficiency or nitrogen availability, and wherein the plant is cultivated in a low nitrogen medium, preferably low nitrogen soil.

44. A method of growing a plant in a low nitrogen medium, said method including infecting a plant with the bioprotectant compound-producing endophyte according to claim 26, and cultivating the plant.

45. The method according to claim 44, wherein the low nitrogen medium is low nitrogen soil.
